(19) United States
(12) Patent Application Publication
(10) Pub. No.: US 2026/0199674 A1
(43) Pub. Date: Jul. 16, 2026

(54) METHODS FOR CONTROLLING SACRAL NEUROMODULATION AND RELATED DEVICES

(71) Applicants: CHINA REHABILITATION SCIENCE INSTITUTE, Beijing (CN); CHINA REHABILITATION RESEARCH CENTER, Beijing (CN)

(72) Inventor: Limin LIAO, Beijing (CN)

(73) Assignees: CHINA REHABILITATION SCIENCE INSTITUTE, Beijing (CN); CHINA REHABILITATION RESEARCH CENTER, Beijing (CN)

(21) Appl. No.: 19/337,893

(22) Filed: Sep. 23, 2025

(30) Foreign Application Priority Data
Jan. 10, 2025 (CN) 202510039622.3

(51) Int. Cl. A61N 1/36

(52) U.S. Cl. CPC A61N 1/36007; A61N 1/36031; A61N 1/36135

(57) ABSTRACT

The present disclosure relates to a method for controlling sacral neuromodulation and related devices. The method includes: obtaining historical physiological parameter information of a target user and real-time physiological parameter information of the target user; processing the real-time physiological parameter information to generate to-be-monitored user data with identification information; obtaining a preset biological neural stimulation signal generation model matching the identification information; processing the preset biological neural stimulation signal generation model based on historical physiological parameter signals to generate a target biological neural stimulation signal generation model; processing the real-time physiological parameter information based on the target biological neural stimulation signal generation model to generate a real-time external anal sphincter electromyogram signal; processing the external anal sphincter electromyogram signal amplitude based on a preset processing rule to generate a sacral neuromodulation signal; and sending the sacral neuromodulation signal to a target terminal device.

### CROSS-REFERENCE TO RELATED APPLICATIONS

This application claims priority to the Chinese Patent Application No. 202510039622.3, filed on Jan. 10, 2025, the contents of which are hereby incorporated by reference.

### TECHNICAL FIELD

The present disclosure relates to the field of data processing, and in particular, to methods for controlling sacral neuromodulation and related devices.

### BACKGROUND

Sacral nerve stimulation or sacral neuromodulation is a treatment for refractory urinary urgency and frequency, with an overactive bladder (OAB) as a representative condition. Sacral neuromodulation modulates bladder function by stimulating the sacral nerves, thereby alleviating symptoms of urinary urgency and frequency. This treatment typically uses an external device to modulate bladder function by electrically stimulating the sacral nerve roots. Current sacral neuromodulation devices employ a continuous stimulation mode. That is, from the time the device is implanted in the patient, and without shutting down, the device continuously sends therapeutic pulses to stimulate the sacral nerves, regardless of whether it is during symptomatic episodes or asymptomatic intervals. This significantly increases power consumption, and a considerable number of patients experience discomfort due to the electrical stimulation. Continuous electrical stimulation increases the negative experience for patients.

Although various machine learning models currently exist for detecting urinary urgency and frequency, large-scale clinical applications remain at a nascent stage. That is, existing detection models are primarily trained based on pre-annotated offline data. This means that existing solutions may only be used to detect urinary urgency and frequency symptoms in the collected data, but cannot perform real-time online detection and intervention. At the same time, the intermittent nature of the urinary urgency and frequency symptoms result in poor detection accuracy and slow detection speed of existing solutions in clinical practice. This makes it difficult for medical staff to perform real-time monitoring and timely intervention through the existing solutions, potentially leading to severe clinical consequences due to the failure to proactively abort urinary urgency and frequency symptoms.

Therefore, the present disclosure provides a method for controlling sacral neuromodulation and related devices. By customizing a biological neural stimulation model, it monitors and generates an external anal sphincter electromyogram signal in real time, and then generates a sacral neuromodulation signal, enabling precise treatment through a target terminal device, continuously optimizing and improving the quality of life for patients.

It should be noted that the information disclosed in the above background art section is only used to enhance the understanding of the background of the present disclosure and therefore may include non-prior art content not known to those of ordinary skill in the art.

### SUMMARY

One or more embodiments of the present disclosure provide a method for controlling sacral neuromodulation, comprising: obtaining historical physiological parameter information of a target user and real-time physiological parameter information of the target user; processing the real-time physiological parameter information of the target user to generate to-be-monitored user data with identification information, wherein the identification information is used to characterize a target item currently to be detected for the target user; obtaining a preset biological neural stimulation signal generation model matching the identification information; processing the preset biological neural stimulation signal generation model based on historical physiological parameter signals of the target user to generate a target biological neural stimulation signal generation model, wherein the historical physiological parameter signals of the target user include training samples corresponding to the target item currently to be detected for the target user; processing the real-time physiological parameter information of the target user based on the target biological neural stimulation signal generation model to generate a real-time external anal sphincter electromyogram signal, wherein the real-time external anal sphincter electromyogram signal includes an external anal sphincter electromyogram signal amplitude, the external anal sphincter electromyogram signal amplitude including a target external anal sphincter diastole amplitude and a target external anal sphincter systole amplitude; and processing the external anal sphincter electromyogram signal amplitude based on a preset processing rule to generate a sacral neuromodulation signal, wherein a preset threshold is set based on the real-time physiological parameter information of the target user.

One or more embodiments of the present disclosure provide an apparatus for controlling sacral neuromodulation, comprising an obtaining module, a processing module, and a sending module. The obtaining module is configured to: obtain historical physiological parameter information of a target user and real-time physiological parameter information of the target user; and obtain a preset biological neural stimulation signal generation model matching identification information. The processing module is configured to process the real-time physiological parameter information of the target user to generate to-be-monitored user data with the identification information. The identification information is used to characterize a target item currently to be detected for the target user. The processing module is further configured to process the preset biological neural stimulation signal generation model based on historical physiological parameter signals of the target user to generate a target biological neural stimulation signal generation model. The historical physiological parameter signals of the target user include training samples corresponding to the target item currently to be detected for the target user. The processing module is further configured to process the real-time physiological parameter information of the target user based on the target biological neural stimulation signal generation model to generate a real-time external anal sphincter electromyogram signal. The real-time external anal sphincter electromyogram signal includes an external anal sphincter electromyogram signal amplitude. The external anal sphincter electromyogram signal amplitude includes a target external anal sphincter diastole amplitude and a target external anal sphincter systole amplitude. The processing module is further configured to process the external anal sphincter electromyogram signal amplitude based on a preset processing rule to generate a sacral neuromodulation signal. A preset threshold is set based on the real-time physiological parameter information of the target user. The sending module is configured to send the sacral neuromodulation signal to a target terminal device, so that the target terminal device operates on the target user.

One or more embodiments of the present disclosure provide an electronic device. The electronic device comprises a first processor and a memory for storing executable instructions of the first processor. The first processor is configured to implement the method for controlling sacral neuromodulation by executing the executable instructions.

One or more embodiments of the present disclosure provide a non-transitory computer-readable storage medium comprising a computer program stored thereon. The computer program, when executed by a second processor, implements the method for controlling sacral neuromodulation.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is an exemplary flowchart of a method for controlling sacral neuromodulation according to some embodiments of the present disclosure;

FIG. 2 is a structural schematic diagram of an apparatus for controlling sacral neuromodulation according to some embodiments of the present disclosure;

FIG. 3 is a structural schematic diagram of an electronic device according to some embodiments of the present disclosure; and

FIG. 4 is a schematic diagram of a storage medium according to some embodiments of the present disclosure.

### DETAILED DESCRIPTION

The accompanying drawings, which are required to be used in the description of the embodiments, are briefly described below. The accompanying drawings do not represent the entirety of the embodiments.

As used herein, “system”, “device”, “unit” and/or “module” is a manner used to distinguish different components, elements, parts, sections, or assemblies at different levels. However, if other words serve the same purpose, the words may be replaced by other expressions.

When describing the operations performed in the embodiments of the present disclosure in step-by-step instructions, the order of the steps is interchangeable if not otherwise specified, the steps are omissible, and other steps may be included in the operation.

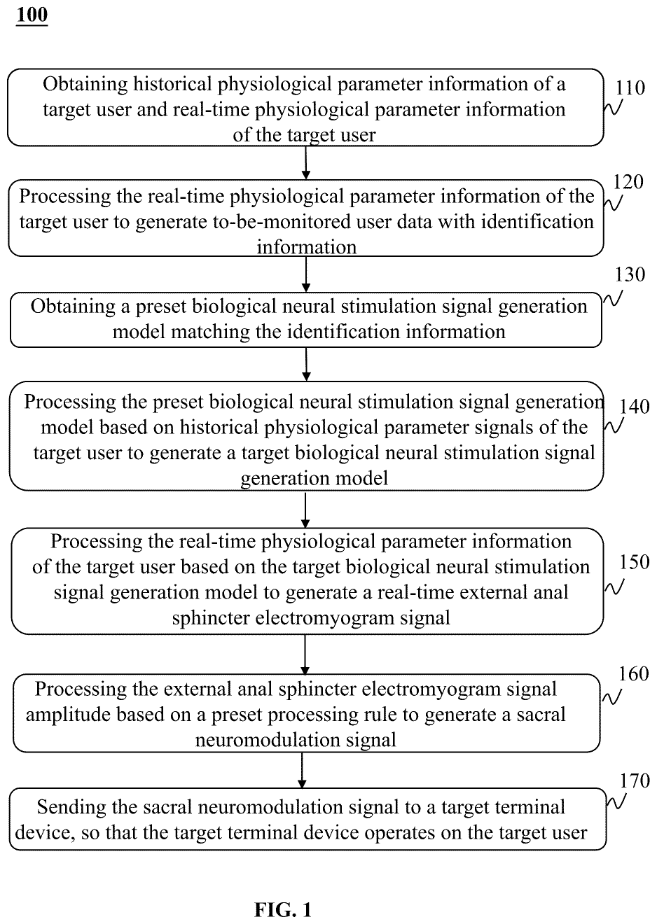

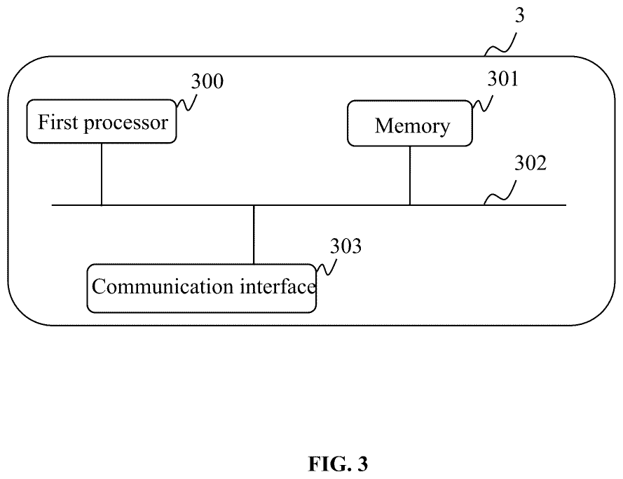

FIG. 1 is an exemplary flowchart of a method for controlling sacral neuromodulation according to some embodiments of the present disclosure. As shown in FIG. 1, process 100 includes the following steps. In some embodiments, the process 100 may be executed by a first processor. For a description of the first processor, refer to the content of FIG. 3 below.

In 110, obtaining historical physiological parameter information of a target user and real-time physiological parameter information of the target user.

The target user refers to a patient receiving sacral neuromodulation treatment, e.g., a patient with urinary urgency symptoms, a patient with an overactive bladder, a patient with bladder dysfunction, etc.

The historical physiological parameter information refers to physiological parameter information collected through historical monitoring.

The real-time physiological parameter information refers to physiological parameter information collected through current real-time monitoring.

The physiological parameter information refers to physiological indicator data related to a physiological state, diseases, etc., of the target user.

The apparatus in the present disclosure may intelligently regulate the timing and intensity of sacral neuromodulation based on a real-time physiological state of the patient with urinary urgency symptoms. To achieve this goal, a series of physiological parameter information needs to be collected and analyzed. In some embodiments, the physiological parameter information includes but is not limited to:

Urodynamic parameters: such as a urinary flow rate, a urine volume, etc., which may reflect a voiding function state of the patient.

Bladder pressure: assess the pressure changes in the bladder during urine storage and voiding through bladder pressure measurement.

Heart rate and blood pressure: provide information about an overall health state of the patient.

Skin electrical activity: reflect the activity of a sympathetic nervous system and may be related to the urinary urgency symptoms.

Abdominal pressure: changes in abdominal pressure may affect bladder pressure and function.

Weight and body mass index (BMI): weight changes may affect the urinary urgency symptoms, and BMI may reflect the patient's body type and health state.

Activity level: data such as steps and activity intensity collected through activity trackers to assess the patient's daily activity patterns.

Sleep quality: such as sleep monitoring data, including sleep cycles, a count of awakenings, etc., which may be related to the occurrence of the urinary urgency symptoms.

Dietary habits: include liquid intake, meal time, etc., as diet may affect a rate of bladder filling and the occurrence of urinary urgency.

Medication use records: medications the patient is taking may affect the voiding function.

Environmental factors: such as temperature, humidity, etc., these factors may indirectly affect the patient's physiological state.

Psychological state indicators: such as a stress level, an emotional state, etc., psychological factors may also affect the urinary urgency symptoms.

In some embodiments, the historical physiological parameter information of the target user may be obtained based on historical records (e.g., hospital patient data records, etc.). The real-time physiological parameter information may be collected in real time through sensors or medical monitoring devices.

In 120, processing the real-time physiological parameter information of the target user to generate to-be-monitored user data with identification information.

The identification information refers to an identifier used to characterize a target item currently to be detected for the target user, such as urinary urgency episode monitoring, etc.

The target item currently to be detected for the target user refers to a physiological indicator that currently requires key monitoring, such as urinary urgency symptom monitoring, etc.

The to-be-monitored user data refers to data that integrates the target item currently to be detected and the identification information of the target user. The to-be-monitored user data includes specific values of the physiological parameter information and key information related to treatment and monitoring.

In some embodiments, the first processor may parse the real-time physiological parameter information of the target user, identify key physiological indicators associated with the target item, input key physiological indicators into a pre-trained machine learning model, output the corresponding identification information, and package the identification information with the target item as the to-be-monitored user data.

In some embodiments, the first processor may also process the real-time physiological parameter information of the target user to generate user attribute information, wherein the user attribute information includes the target item currently to be detected for the target user; perform feature extraction on the real-time physiological parameter information of the target user to generate a target feature library; process the user attribute information based on the target feature library to generate the to-be-monitored user data with the identification information.

The user attribute information refers to characteristic information of the user generated after processing the real-time physiological parameter information, including the target item to be detected for the target user, such as the urinary urgency symptom monitoring, etc.

Collect the patient's physiological parameter information in real time, such as bladder pressure, urinary flow rate, heart rate, blood pressure, etc, and these parameters are crucial for assessing the urinary urgency symptoms. Process the collected real-time physiological parameter information to generate the to-be-monitored user data with the identification information, and the identification information clearly indicates the target item that currently needs to be detected and focused on.

In some embodiments, the first processor may extract key data points from the real-time physiological parameter information to generate the user attribute information. The user attribute information is directly linked to specific medical goals to be detected for the patient, such as the severity of the urinary urgency symptoms.

The target feature library refers to a dataset generated by performing feature extraction on the real-time physiological parameter information of the target user.

In some embodiments, the first processor may perform in-depth analysis on the user attribute information, extract features that may characterize the patient's condition through feature extraction techniques (e.g., time-domain analysis, frequency-domain analysis, etc.), and build the target feature library. These features include urinary flow patterns and specific patterns of pressure changes.

The urinary flow patterns refer to information about urine flow changing over time, including but not limited to the urinary flow rate, a voiding time, a uroflow curve, and a maximum flow rate. The urinary flow rate refers to a volume of urine expelled per unit time, usually expressed in milliliters/second, and normal flow rates vary with age, gender, and urine volume. The voiding time refers to a total time required to complete urination. The uroflow curve refers to a curve formed by measuring the change in urine flow speed over time with a uroflowmeter, which may reflect the voiding function of the target user. The maximum flow rate refers to a maximum value in the uroflow curve and is a key indicator for evaluating voiding dysfunction.

The specific patterns of pressure changes refer to characteristic changes reflecting bladder and urethral function through the measurement and analysis of urodynamic pressure parameters, including but not limited to cystometry, a detrusor pressure, a urethral closure pressure, and a pressure-flow relationship. The cystometry refers to measuring pressure changes in the bladder during urine storage and voiding using a cystometer, which may reflect bladder compliance and emptying ability. The detrusor pressure refers to a contraction pressure of the detrusor muscle during voiding, which may reflect bladder emptying efficiency. The urethral closure pressure refers to a closure pressure of the urethral sphincter during urine storage, which may be used to assess urethral closure function. The pressure-flow relationship refers to a relationship between the bladder pressure and the urinary flow rate, which may reveal dynamic changes during the voiding process.

In some embodiments, the urinary flow patterns and the specific patterns of pressure changes are crucial for diagnosing a cause of the urinary urgency symptoms (e.g., bladder overactivity, urethral stricture, etc.). For example, urinary urgency may be accompanied by rapidly increasing the bladder pressure and the urinary flow rate, reflecting bladder overactivity or sensory hypersensitivity. The urinary urgency may cause the uroflow curve to show rapid rise and fall, indicating difficulty in controlling urination.

In some embodiments of the present disclosure, based on the parameter measurement results of the urinary flow patterns and the specific patterns of pressure changes, doctors can formulate personalized treatment plans, such as medication, biofeedback, neuromodulation, or surgery. The target feature library is used to further process the user attribute information to enrich and refine user data, ensuring data integrity and accuracy.

In some embodiments, the first processor may process the user attribute information based on the target feature library to generate the to-be-monitored user data with the identification information. For example, the first processor associates abnormal patterns in the target feature library (e.g., a sharp rise in bladder pressure+a sudden drop in the urinary flow rate matching a urinary urgency episodepattern) with the target item in the user attribute information (e.g., the urinary urgency symptom monitoring) to generate the to-be-monitored user data with the identification information (e.g., the urinary urgency episode monitoring).

In some embodiments of the present disclosure, by combining the feature library processing results and the user attribute information, the final to-be-monitored user data with the identification information is generated, i.e., data containing the specific values of the physiological parameters and all key information related to treatment and monitoring. Subsequently, by sending the generated signals to the target terminal device to guide the device in performing precise operations on the patient, the therapeutic effect can be maximized. This not only improves the precision and personalization level of treatment but also provides a more efficient and comfortable treatment experience for the patient with urinary urgency symptoms through intelligent data processing and feature extraction. At the same time, it also helps reduce medical costs and improve the utilization efficiency of medical resources.

In 130, obtaining a preset biological neural stimulation signal generation model matching the identification informationis.

The preset biological neural stimulation signal generation model refers to a biological neural stimulation signal generation model obtained through pre-training, such as a model generated by pre-training a machine learning model (e.g., support vector machine (SVM), a neural network, etc.,) using historical physiological parameter data and historical identification information of historical patients collected from a large number of clinical experiments as training samples, and historical actual stimulation parameters as training labels.

In some embodiments, the identification information characterizes urinary urgency symptom-related items currently to be detected for the target user, such as urinary flow patterns, bladder pressure, etc. A preset model library containing a plurality of preset biological neural stimulation signal generation models is established. These models are designed for different physiological parameters and symptoms. Based on the identification information, the most suitable preset biological neural stimulation signal generation model is matched from the preset model library. The matching process may involve corresponding algorithms, such as decision trees, random forests, gradient boosting machines, neural networks, or other machine learning algorithms, which are not limited in the present disclosure.

In 140, processing the preset biological neural stimulation signal generation model based on historical physiological parameter signals of the target user to generate a target biological neural stimulation signal generation model.

The historical physiological parameter signals are the historical physiological parameter information.

The target biological neural stimulation signal generation model refers to a biological neural stimulation signal generation model personalized and adjusted based on the physiological parameters of the target user.

An input of the target biological neural stimulation signal generation model is the real-time physiological parameter information of the target user, and an output is the sacral neuromodulation signal (including frequency, pulse width, intensity parameters).

The historical physiological parameter signals of the target user are the historical physiological parameter information of the target user, including training samples corresponding to the target item currently to be detected for the target user.

The training samples are historical data extracted from the historical physiological parameter signals of the target user and directly related to a current target item to be detected. For example, if the current target item to be detected is urinary urgency suppression response, the corresponding training samples only use electromyogram signal segments in the historical physiological parameter signals labeled as successful/failed urinary urgency suppression.

In some embodiments, the first processor may analyze the historical physiological parameter signals of the target user to obtain target key parameters (e.g., a baseline value, a contraction value, etc.), and use them to replace the corresponding preset key parameters in the preset biological neural stimulation signal generation model to generate the target biological neural stimulation signal generation model. The baseline value is a maximum value of a target external anal sphincter diastole amplitude in historical external anal sphincter electromyogram signals, and the contraction value is a maximum value of a target external anal sphincter systole amplitude in historical signals.

By analyzing the historical physiological parameter information of the target user, understanding the long-term trends and characteristics of their symptoms, and combining user historical data and real-time data, the preset biological neural stimulation signal generation model is personalized and adjusted to adapt to the specific user's physiological state and feature parameters such as urinary flow patterns and pressure changes are integrated into the model to ensure that the generated signals may be optimized for these key indicators. Before the practical application of the model, the model's effectiveness and safety are validated through simulation or small-scale clinical trials. During model application, monitor the user's physiological response in real time and collect feedback information for dynamic model adjustment. Then, the adjusted model is used to generate the sacral neuromodulation signal based on the user's real-time physiological parameters, and the parameters of the stimulation signal (e.g., the pulse width, the frequency, the intensity, etc.) are continuously optimized based on user feedback and treatment effects. Finally, the generated biological neural stimulation signal is applied to clinical treatment, implementing precise stimulation through the target terminal device.

In some embodiments of the present disclosure, all relevant data during the treatment process is recorded and analyzed in depth to further improve the model's accuracy and therapeutic effect. This ensures that each patient receives personalized nerve stimulation therapy, improves the treatment effect for the urinary urgency symptoms, reduces unnecessary stimulation, and enhances the patient's quality of life.

In some embodiments, the first processor may also perform feature extraction on the historical physiological parameter signals of the target user to generate an original feature library, wherein the original feature library includes discrete variables and continuous variables; process the historical physiological parameter signals of the target user based on the discrete variables to generate an initial training set; process the initial training set based on the continuous variables to generate a target training set, wherein the target training set includes a plurality of external anal sphincter electromyogram signal amplitude reference intervals.

The original feature library refers to a structured dataset generated by performing the feature extraction on the historical physiological parameter signals. The original feature library includes the discrete variables and the continuous variables.

The discrete variables refers to a collection of discrete features extracted from the historical physiological parameter signals for classification. For example, the discrete variables includes categorical labels such as gender (male/female), disease type (overactive bladder/neurogenic bladder), medication use (yes/no), etc.

The continuous variables refers to a collection of features extracted from the historical physiological parameter signals that is measured numerically, e.g., age, a bladder pressure value, the urinary flow rate, the external anal sphincter electromyogram signal amplitude, etc.

In some embodiments, the physiological parameter signals of the target user are collected at different time points. These signals correspond to the urinary urgency symptom-related items currently to be detected. The collected signals are analyzed and features that may characterize the user's physiological state (e.g., a peak urinary flow rate, the bladder pressure changes, etc.) are extracted. The extracted features are classified into the discrete variables (e.g., gender, the disease type) and the continuous variables (e.g., age, the urinary flow rate, the pressure values) to build the original feature library.

The initial training set is a dataset generated by classifying the historical physiological parameter signals based on the discrete variables.

In some embodiments, the first processor may use the discrete variables in the original feature library to classify and group the historical physiological parameter signals, generating the initial training set.

The target training set is a dataset generated by performing cluster analysis on the initial training set based on the continuous variables, including a plurality of external anal sphincter electromyogram signal amplitude reference intervals.

In some embodiments, the first processor may combine the processing results of the discrete variables and the continuous variables to generate the target training set. This dataset will be used to establish reference intervals for the external anal sphincter electromyogram signal. For example, the first processor calculates, based on the continuous variables in the initial training set, electromyogram amplitude boundary values through cluster analysis (e.g., K-means), generates the reference intervals for the external anal sphincter electromyogram signal amplitude of the target user according to an amplitude distribution (e.g., diastole [80-100 μV], systole [120-150 μV]), and constitutes the target training set.

In some embodiments, the first processor may determine the external anal sphincter electromyogram signal amplitude reference intervals based on the target training set. These intervals will serve as the basis for assessing the patient's current state and generating stimulation signals. Use the target training set to train the biological neural stimulation signal generation model, enabling the biological neural stimulation signal generation model to predict optimal stimulation parameters based on the physiological parameter signals.

The reference interval for the external anal sphincter electromyogram signal refers to a effective signal range dynamically calculated based on the external anal sphincter electromyogram signal amplitude reference interval and the real-time physiological parameter information, used to determine whether the physiological state is in a urinary urgency suppression period.

The external anal sphincter electromyogram signal amplitude reference interval refers to a static numerical range extracted from historical continuous variables, characterizing a normal distribution range of the external anal sphincter electromyogram signal amplitude. The external anal sphincter electromyogram signal amplitude reference interval may serve as the basis for assessing the patient's current state and generating stimulation signals.

In some embodiments of the present disclosure, screening the initial training set with the discrete variables ensures that the model focuses on a specific pathological state of the target user, improving model personalization accuracy. Using the continuous variables to generate personalized reference intervals provides precise input for dynamic threshold calculation, enabling on-demand stimulation, which is beneficial for optimizing treatment real-time performance.

In some embodiments, the first processor may process the target training set to generate multiple continuous variables matching the identification information; process the multiple continuous variables based on a preset function to generate a target external anal sphincter electromyogram signal amplitude reference interval, wherein the target external anal sphincter electromyogram signal amplitude reference interval is a preset reference range matching the target user; generate the target biological neural stimulation signal generation model based on the target external anal sphincter electromyogram signal amplitude reference interval.

The multiple continuous variables matching the identification information refers to a combination of a plurality of quantifiable physiological parameters corresponding to the target item to be monitored, for example, multiple key continuous variables affecting the external anal sphincter electromyogram signal amplitude.

In some embodiments, the first processor may screen continuous variables corresponding to the target event in the target training set based on the identification information, and integrate them into the multiple continuous variables matching the identification information.

In some embodiments, the multiple key continuous variables affecting the external anal sphincter electromyogram signal amplitude include the urinary flow rate, the bladder pressure, the electrical activity signal of the external anal sphincter, coordination between the detrusor and the external anal sphincter, etc.

The urinary flow rate is a key indicator for measuring voiding efficiency. Analyzing trends in the urinary flow rate (e.g., the maximum flow rate, the average flow rate, and the voiding time) may reveal the state of the voiding function. The bladder pressure measurement provides dynamic information about the bladder during urine storage and voiding. Monitoring changes in the bladder pressure helps assess bladder compliance and emptying ability. The electrical activity signal of the external anal sphincter reflects the tension and coordination of the sphincter. Analyzing the amplitude and pattern of the external anal sphincter's electrical activity signal may reveal its functional state. The coordination between the detrusor and the external anal sphincter is crucial for voiding control. Analyzing an interaction between the detrusor and the external anal sphincter may identify coordination disorders. Therefore, identifying and processing the multiple key continuous variables affecting the external anal sphincter electromyogram signal amplitude is an important component of achieving precise medical intervention.

In some embodiments, the first processor may analyze the multiple key continuous variables affecting the external anal sphincter electromyogram signal amplitude in various ways. For example, perform correlation analysis on parameters such as the urinary flow rate, the bladder pressure, and the external anal sphincter electromyogram to identify their interactions and dependencies; apply time series analysis manners to study the change patterns of physiological parameters over time to predict the occurrence and intensity of the urinary urgency symptoms; use machine learning techniques, such as support vector machines, random forests, or the neural networks, to model the multiple key continuous variables to identify key factors affecting the external anal sphincter electromyogram signal amplitude.

The preset function refers to a statistical function used to calculate personalized reference intervals. For example, the target external anal sphincter electromyogram signal amplitude reference interval=μ+kσ, μ is a mean of the external anal sphincter electromyogram signal amplitude, σ is a standard deviation, and k is an age adjustment coefficient.

The target external anal sphincter electromyogram signal amplitude reference interval refers to a reference interval for the external anal sphincter electromyogram signal amplitude exclusive to the target user, distinct from universal population reference intervals, used for accurately determining the functional state of the sphincter for the target user.

In some embodiments, the first processor may apply the preset function to analyze selected features and generate the target external anal sphincter electromyogram signal amplitude reference interval. The generated reference interval defines normal and abnormal physiological states. For example, the first processor may substitute the multiple continuous variables into the preset function to generate the reference interval. Exemplarily, for elderly patients: set k=1.5, calculate μ±1.5×σ.

In some embodiments, the first processor may implant the baseline value (maximum diastole amplitude) and the contraction value (maximum systole amplitude) from the target external anal sphincter electromyogram signal amplitude reference interval into the preset biological neural stimulation signal generation model, updating a trigger threshold formula to generate the target biological neural stimulation signal generation model. The trigger threshold T=the baseline value+the trigger coefficient×(the contraction value—the baseline value).

In some embodiments, the target biological neural stimulation signal generation model takes the historical physiological parameters of the target user and real-time collected physiological data as input, sets stimulation parameters and trigger parameters, calculates the trigger threshold, takes the target external anal sphincter electromyogram signal amplitude reference interval and the multiple continuous variables matching the identification information as input, and uses the preset function to determine the physiological state. When real-time parameters meet a trigger condition (e.g., real-time electromyogram amplitude exceeds the trigger threshold), it outputs a personalized sacral neuromodulation signal containing electrical stimulation frequency, the pulse width, the intensity, and a single stimulation duration.

The trigger parameters include the baseline value, the contraction value, and the trigger coefficient. The baseline value is a maximum electromyogram amplitude during diastole; the contraction value is a maximum contraction electromyogram amplitude during systole; the trigger coefficient is a parameter in a trigger threshold calculation, which may be adjusted based on triggering situations, initially set to 50%. The stimulation parameters may be set as follows: electrical stimulation frequency is generally 14 Hz, pulse width is 210 μs; the stimulation intensity is determined based on intraoperative motor response and sensory response, generally set slightly below a threshold intensity of the motor responses and the sensory responses, and adjusted postoperatively based on effect; the single stimulation duration is 1 minute and is subsequently adjusted based on effect.

In some embodiments of the present disclosure, by applying the preset function to analyze selected features and generate the target external anal sphincter electromyogram signal amplitude reference interval, it ensures that the generated reference interval matches the specific physiological characteristics and medical history of the target user, enabling personalized assessment of the target user. Through the adjusted and optimized target biological neural stimulation signal generation model, appropriate stimulation signals can be generated based on the patient's real-time physiological state. Through this model, highly personalized and precise treatment plans can be provided for patients with urinary urgency symptoms, optimizing the generation of biological neural stimulation signals, improving treatment efficacy, reducing unnecessary stimulation, and enhancing the patient's quality of life.

In some embodiments, the training samples for the biological neural stimulation signal generation model include a plurality of groups, each group corresponding to different training features, and the plurality of groups of the training samples are grouped based on the training features. Additionally, the first processor may adjust the learning rate corresponding to the training samples during model training based on the training features.

The training features may include an age group, gender, symptom type, symptom duration, etc.

The symptom duration refers to a cumulative duration of symptoms, such as urinary urgency and frequency, experienced by the patient.

In some embodiments, the first processor may group the training samples into the plurality of groups based on combinations of the training features, each group corresponding to one combination of the training features.

In some embodiments, the first processor may establish a first preset table based on the training features, and then determine the learning rate for training the model with each group of the training samples by querying the first preset table. The first preset table includes the correspondence between training feature combinations and the learning rates. For example, the higher the data sample density of the training feature combination and the higher the stimulation effectiveness, the lower the corresponding learning rate.

The data sample density refers to a proportion of the training samples for a particular training feature combination within all training samples. The stimulation effectiveness refers to a proportion by which the user's symptom relief degree reaches an expected treatment goal under the sacral neuromodulation. For example, if urinary urgency frequency decreases by 20% after stimulation, and the expected decrease is 80%, then the stimulation effectiveness is 25%.

In some embodiments, the first preset table may be constructed based on treatment records of historical users with good treatment effects (e.g., the stimulation effectiveness ≥80%). For example, the first preset table is constructed based on a large number of treatment records of the historical users with good treatment effects. Each record includes the user's training features and the stimulation parameters of an actually selected sacral neuromodulation signal. Fill a table with the data to build the first preset table.

In some embodiments, the target biological neural stimulation signal generation model may be trained in various ways. The training process may include a transfer learning process and a reinforcement learning process.

In some embodiments, the transfer learning process includes: performing the feature extraction on the historical physiological parameter signals of the target user to generate the original feature library; generating the initial training set based on the discrete variables; generating the target training set based on the continuous variables; generating the target external anal sphincter electromyogram signal amplitude reference interval based on the target training set; generating the target biological neural stimulation signal generation model based on the target external anal sphincter electromyogram signal amplitude reference interval.

In some embodiments, the reinforcement learning process includes: after the training of the target biological neural stimulation signal generation model is completed, the first processor may personalize and adjust the target biological neural stimulation signal generation model based on real-time feedback data of the target user. The real-time feedback data refers to dynamic data collected for the target user during the execution of sacral neuromodulation that reflects the physiological response state and symptom changes of the target user, which may include a electromyogram of the external anal sphincter signal, sacral neuromodulation signal parameters, the stimulation effectiveness, etc.

By continuously evaluating the stimulation effect (e.g., external anal sphincter electromyogram response, symptom relief, etc.) and using it as feedback signals, the model parameters are iteratively optimized, thereby achieving more precise stimulation signal output.

In some embodiments of the present disclosure, by grouping the training samples according to the training features such as age group, gender, symptom type, symptom duration, and adjusting the learning rate during the model training based on the training features, fine-grained management of the training samples and adaptive optimization of model parameters are achieved, improving the reliability of the output results of the target biological neural stimulation signal generation model.

In 150, processing the physiological parameter information of the target user based on the target biological neural stimulation signal generation model to generate a real-time external anal sphincter electromyogram signal.

The real-time external anal sphincter electromyogram signal refers to an original waveform signal reflecting the electrical activity of the external anal sphincter, collected in real time by an electromyography monitoring module, including the external anal sphincter electromyogram signal amplitude.

The external anal sphincter electromyogram signal amplitude refers to a physical magnitude value of the external anal sphincter electromyogram signal waveform, including the target external anal sphincter diastole amplitude and the target external anal sphincter systole amplitude.

The target external anal sphincter diastole amplitude refers to a maximum electromyogram amplitude value reached by the sphincter in a resting state, i.e., the maximum electromyogram amplitude during diastole, used to calculate the baseline value of the trigger threshold.

The target external anal sphincter systole amplitude refers to the maximum electromyogram amplitude value reached by the sphincter in a contracted state, i.e., the maximum contraction electromyogram amplitude during systole, used to calculate the contraction value of the trigger threshold.

In some embodiments, the first processor may determine the maximum electromyogram amplitude during diastole and the maximum contraction electromyogram amplitude during systole by repeatly collecting electromyography signals from the external anal sphincter during diastole and systole. The trigger threshold is determined based on the maximum electromyogram amplitude during diastole and the maximum contraction electromyogram amplitude during systole, and the trigger threshold lies between the maximum electromyogram amplitude during diastole and the maximum contraction electromyogram amplitude during systole.

In some embodiments, the first processor may input the physiological parameter information of the target user into the target biological neural stimulation signal generation model, extract frequency band components through Fourier transform, and calculate power spectral entropy and sample entropy; output the real-time external anal sphincter electromyogram signal based on a frequency band energy distribution and an entropy result.

In some embodiments, the first processor may process the target training set based on the target biological neural stimulation signal generation model to generate a preset external anal sphincter electromyogram signal dataset; process the preset external anal sphincter electromyogram signal dataset to obtain stimulation features and a correlation co-occurrence frequency; process the stimulation features and the correlation co-occurrence frequency to generate correlation matrix information; generate a prior knowledge graph for characterizing the real-time external anal sphincter electromyogram signal based on the correlation matrix information; process the physiological parameter information of the target user based on the prior knowledge graph to generate the real-time external anal sphincter electromyogram signal.

The preset external anal sphincter electromyogram signal dataset refers to a standardized dataset containing historical electromyogram signal feature vectors, generated by processing the target training set with the target biological neural stimulation signal generation model. It is used to provide prior knowledge for the initial training of the biological neural stimulation model.

In some embodiments, the first processor may input the target training set into the target biological neural stimulation signal generation model and output the preset external anal sphincter electromyogram signal dataset.

The stimulation features refer to quantifiable electrical signal parameters that directly trigger or are associated with the generation of the sacral neuromodulation signal, e.g., an electromyogram signal amplitude, a duration, and a change pattern.

The correlation co-occurrence frequency refers to a conditional probability that conceptual features (e.g., bladder pressure and sphincter electromyogram) co-occur in the dataset.

In some embodiments, the first processor may apply the target biological neural stimulation signal generation model to process the target training set and generate the preset external anal sphincter electromyogram signal dataset, analyze the generated preset external anal sphincter electromyogram signal dataset to identify the stimulation features, such as the amplitude, duration, and change pattern of the electromyogram signal, count a co-occurrence count of each feature pair and a total occurrence count of the target feature in report-level events, and obtain the correlation co-occurrence frequency through a formula for calculating co-occurrence frequency. The report-level event refers to a structured data record unit generated during a single complete urinary urgency symptom medical monitoring event.

The correlation matrix information refers to a 0/1 matrix characterizing binary correlation relationships between stimulation features.

In some embodiments, the first processor may process the generated preset external anal sphincter electromyogram signal dataset in various ways to obtain the correlation co-occurrence frequency and correlation matrix. For example, by building the machine learning model, count the co-occurrence frequency of various parameters in the time series to obtain the correlation co-occurrence frequency; then standardize the data, determine the degree of association for each parameter pair through a correlation calculation method and map it to matrix elements; finally build matrix information containing correlations between physiological features, between stimulation parameters, and between the two types of parameters. In some embodiments, the first processor calculates the correlation co-occurrence frequency between the stimulation features to identify their interrelationships and occurrence patterns.

In some embodiments, the method for controlling sacral neuromodulation includes a first calculation formula for calculating the correlation co-occurrence frequency, the first calculation formula being:

$${P}_{ij} = \frac{{C}_{ij}}{{C}_{j}}$$

where, Cᵢⱼ represents a count of times concept i and concept j co-occur at a report level, Cⱼ represents a total count of times concept j appears, and Pᵢⱼ represents a frequency of concept i appearing when concept j appears.

The count of times concept i and concept j co-occur at the report level refers to a total count of times the concept i and the concept j appear together in the same physiological monitoring report. The first processor may traverse all historical monitoring reports and count a co-occurrence frequency of a concept pair (i, j) in the same report as Cᵢⱼ. The total count Cⱼ of times the concept j appears refers to a total count of times the concept j appears independently in historical monitoring reports. The frequency Pᵢⱼ of the concept i appearing when the concept j appears refers to the conditional probability or frequency proportion of the concept i appearing simultaneously when the concept j appears independently in historical monitoring reports. The frequency Pᵢⱼ of the concept i appearing when the concept j appears is obtained by calculating the ratio of Cᵢⱼ to Cⱼ.

The method for controlling sacral neuromodulation further includes a second calculation formula for calculating the correlation matrix information, the second calculation formula being:

$${A}_{ij} = \{ \begin{matrix}1 , & if {P}_{ij} \geq τ \\ 0 , & if {P}_{ij} < τ\end{matrix}$$

where, Aᵢⱼ represents the correlation matrix information, τ represents a co-occurrence frequency threshold, and if Pᵢⱼ is greater than or equal to τ, it is considered that there is a correlation from the concept i to the concept j, otherwise there is no correlation.

The co-occurrence frequency threshold τ refers to a preset critical probability value for determining whether there is a correlation between concepts, set by system default or predefined manually.

In some embodiments of the present disclosure, the first calculation formula for the correlation co-occurrence frequency quantifies feature association strength, accurately reflecting dependencies between physiological parameters. The second calculation formula for the correlation matrix determines whether there is a correlation between features, is used to filter out noise information, thus focusing on key features that have a practical impact on sacral neuromodulation, and avoiding interference from invalid features.

The prior knowledge graph refers to a directed graph structure with the stimulation features as nodes and correlations as edges. The graph provides an intuitive understanding of relationships between signal features and is used to describe physiological association rules of the electromyogram signals. The direction of the edge indicates a physiological influence path of the stimulation features.

In some embodiments, the first processor may utilize the correlation matrix information to map the correlation matrix Aᵢⱼ into a graph structure, i.e., the prior knowledge graph. For example, if Aᵢⱼ=1, then there is an edge from node i to node j.

In some embodiments, a processing module may apply rules and patterns from the prior knowledge graph to generate the real-time external anal sphincter electromyogram signal matching the current physiological state of the target user. At the same time, the processing module may adjust the parameters of the biological neural stimulation signal generation model based on real-time signal features and the prior knowledge graph to optimize the stimulation effect.

In some embodiments of the present disclosure, by processing the target training set to generate the preset external anal sphincter electromyogram signal dataset and extracting stimulation features and correlation co-occurrence frequency from it, the accuracy of signal generation is improved, ensuring the generated electromyogram signals fit the real physiological state. By generating the correlation matrix information and constructing the prior knowledge graph, the model's dynamic adaptability to individual physiological characteristics is enhanced, providing support for personalized treatment. By processing physiological parameters in real time based on the prior knowledge graph and generating the electromyogram signals, the problem of poor real-time performance of existing offline models is solved, achieving on-demand stimulation to reduce discomfort and power consumption.

In 160, processing the external anal sphincter electromyogram signal amplitude based on a preset processing rule to generate a sacral neuromodulation signal.

The sacral neuromodulation signal refers to a specific electrical pulse waveform generated by a pulse generator module for suppressing urinary urgency episodes. Core parameters include: frequency of 14 Hz (default, adjustable), pulse width of 210 μs (default, adjustable), intensity of slightly below a threshold intensity of intraoperative motor and sensory response.

The preset processing rule refers to predefined signal processing and stimulation generation criteria, which may be set by experts based on clinical experience, such as the trigger threshold formula mentioned above.

The preset threshold refers to a threshold used to judge the external anal sphincter electromyogram signal amplitude and determine whether to trigger the sacral neuromodulation signal, i.e., the trigger threshold. The preset threshold may be set based on the real-time physiological parameter information of the target user.

In some embodiments, the first processor may process the real-time external anal sphincter electromyogram signal to generate feature information, and analyze and process the feature information using data analysis algorithms (e.g., feature vector extraction algorithms, pattern recognition algorithms, etc.), generate a judgment result based on the external anal sphincter electromyogram signal amplitude. If the judgment result indicates that the electromyogram signal amplitude is greater than the preset threshold, the first processor may generate the sacral neuromodulation signal; if the judgment result indicates that the electromyogram signal amplitude is not greater than the preset threshold, the first processor may maintain a monitoring state.

In some embodiments, the first processor may process the real-time external anal sphincter electromyogram signal to generate an electromyogram signal feature vector; process the electromyogram signal feature vector based on the target biological neural stimulation signal generation model to generate a classification result, wherein the classification result is set based on the external anal sphincter electromyogram signal amplitude; if the classification result is that the external anal sphincter electromyogram signal amplitude is greater than the preset threshold, generate the sacral neuromodulation signal.

The electromyogram signal feature vector refers to a multi-dimensional data vector extracted after performing operations such as filtering and windowing processing, Fourier transform, and power calculation on the real-time external anal sphincter electromyogram signal, which may characterize a time-domain feature and a frequency-domain feature of the signal.

In some embodiments, the first processor may perform filtering and windowing processing on the real-time external anal sphincter electromyogram signal, segment the filtered signal into a plurality of time windows for time-frequency analysis, obtain a plurality of frequency band signals, perform Fourier transform on the signal of each time window to extract signal components of different frequency bands, perform power calculation processing on the plurality of frequency band signals to obtain a plurality of power values, calculate the power spectral entropy and the sample entropy of the real-time external anal sphincter electromyogram signal, obtain the electromyogram signal feature vector based on the power spectral entropy, sample entropy, and the plurality of power values. The first processor may use the power values to calculate the power spectral entropy, and combine the power spectral entropy, the sample entropy, and the plurality of power values to construct the electromyogram signal feature vector.

The feature vector contains key features of the signal and may be used for pattern recognition and classification. Standardizing the feature vector may eliminate the dimensional influence between different features. Necessary preprocessing is performed on the collected signal, including filtering, denoising, etc., to improve signal quality. The key features of the electromyogram signal (e.g., amplitude, frequency, duration, etc.) are extracted to generate the electromyogram signal feature vector. The power spectral entropy is an indicator that quantifies the complexity of the signal's frequency distribution.

The classification result refers to a binary decision label generated based on the comparison between the external anal sphincter electromyogram signal amplitude and the preset threshold.

In some embodiments, the classification result is set based on the external anal sphincter electromyogram signal amplitude. The first processor may generate a classification based on the external anal sphincter electromyogram signal amplitude according to the model's analysis results. The classification may include “normal”, “abnormal”, or specific physiological state identifiers. In some embodiments, the first processor may also dynamically set one or more thresholds based on the real-time physiological parameter information of the target user to determine whether the electromyogram signal amplitude exceeds a normal range.

In some embodiments, if the classification result is that the external anal sphincter electromyogram signal amplitude is greater than the preset threshold, the first processor may generate the sacral neuromodulation signal. The preset threshold is set based on the real-time physiological parameter information of the target user. When a patient experiences the urinary urgency episode, the patient actively contract the anus, increasing the external anal sphincter electromyogram amplitude. The receiving electrode picks up the signal and transmits it to the electromyography monitoring module. After filtering, an electromyogram amplitude value is extracted in real time and transmitted to a control module. The first processor determines that the transmitted electromyogram amplitude is greater than the set trigger threshold, activates the pulse generator module to deliver a brief electrical pulse to the sacral nerve roots, suppressing the urinary urgency episode.

In some embodiments of the present disclosure, by processing the real-time external anal sphincter electromyogram signal to generate a feature vector and using the target biological neural stimulation signal generation model to classify and analyze the feature vector, the electromyogram signal amplitude is precisely assessed, ensuring that the classification result accurately reflects the functional state of the external anal sphincter. By dynamically setting the preset threshold based on the real-time physiological parameter information of the target user, personalized adaptation of stimulation trigger conditions is achieved, avoiding misjudgments or missed judgments caused by individual differences with traditional fixed thresholds. By combining the classification result with the dynamic threshold to judge and generate the sacral neuromodulation signal, precise “on-demand stimulation” intervention is realized. This effectively improves the patient's adaptability to voltage, alleviates the discomfort felt by the patient each time the stimulator initiates pulse stimulation, enhances the real-time suppression effect on the urinary urgency episode, and solves the problems of rigid stimulation patterns and poor real-time performance in conventional technical solutions.

In some embodiments, to reduce the issue in conventional technical solutions where the target voltage of the neuromodulation is set directly upon startup, causing the patient to feel intense stimulation without warning and affecting patient comfort, the processing module, upon receiving the sacral neuromodulation signal, may make a single stimulation consist of a plurality of pulse waveforms at a fixed frequency. The first pulse waveform has a voltage that rapidly rises from 0 to the target voltage value of the patient in the first half, and subsequent pulse waveforms are all square wave pulses at the target voltage.

In some embodiments, the method for controlling sacral neuromodulation further includes a third calculation formula for generating the voltage rise of the first pulse upon each startup, specifically as follows:

$$f ( t ) = \{ \begin{matrix}A ( 1 - {e}^{- Bt} ) & if 0 \leq t \leq 100 μs \\ A & if 100 μs < t \leq 200 μs\end{matrix}$$

where, A is a target voltage value of a delivered pulse, B is a rise rate constant, defaulting to 100, and the speed of voltage rise may be adjusted by changing the value of B.

The target voltage value of the delivered pulse refers to a maximum voltage value after the pulse stabilizes, set based on the threshold intensity of the intraoperative motor and sensory responses. The rise rate constant B controls the exponential rate at which the voltage rises from 0 to A, set by the system default to 100.

In some embodiments of the present disclosure, by integrating the real-time physiological parameter information and the historical physiological parameter information, the system can generate the to-be-monitored user data with the identification information, providing personalized treatment plans for patients. At the same time, it not only helps assess the patient's current condition but can also be used for long-term tracking of treatment effects and adjustment of treatment plans. By obtaining the historical physiological parameters and the real-time physiological parameters of the target user, more precise and effective treatment can be provided while improving the patient's treatment experience. By matching the preset biological neural stimulation signal generation model and optimizing the generation of the target biological neural stimulation signal generation model based on historical parameters, personalized adaptation of stimulation strategies is achieved. Based on the patient's urinary urgency symptoms, the timing of sacral nerve electrical pulse delivery is controlled through active external anal sphincter contraction, achieving true “on-demand stimulation”. This avoids the discomfort caused by continuous stimulation, reduces power consumption, extends device usage cycles, alleviates economic burdens, improves the treatment experience, reduces medical economic burdens, and has high medical utility value.

In 170, sending the sacral neuromodulation signal to a target terminal device, so that the target terminal device operates on the target user.

The target terminal device refers to a medical device that receives the sacral neuromodulation signal and performs neuromodulation operations on the target user, which may be an implantable sacral neuromodulation device, an external wearable neuromodulation device, a remotely controllable intelligent stimulation terminal, etc.

In some embodiments, the target terminal device collects the external anal sphincter electromyogram signal of the target user in real time through surface or implanted electrodes, transmits the collected signal to the electromyography monitoring module for filtering and windowing preprocessing, performs Fourier transform on the preprocessed signal to extract power values of signals in different frequency bands, calculates the power spectral entropy and the sample entropy of the real-time external anal sphincter electromyogram signal, constructs the electromyogram signal feature vector based on the power spectral entropy, the sample entropy, and the plurality power values, and inputs the feature vector into the target biological neural stimulation signal generation model to generate the classification result.

The preset threshold is dynamically set based on the real-time physiological parameter information of the target user. When the real-time monitored external anal sphincter electromyogram signal amplitude exceeds the dynamically set preset threshold and the model-generated classification result is abnormal, the first processor, according to a rule where the first pulse voltage rises progressively from 0 to the target value following an exponential function based on the third calculation formula for the first pulse upon each startup, and subsequent pulses are square waves, generates the sacral neuromodulation signal and sends it to the target terminal device. The target terminal device, upon receiving the sacral neuromodulation signal, applies it to the sacral nerve roots to perform neuromodulation. At the same time, the first processor optimizes the parameters of the target biological neural stimulation signal generation model based on stimulation effect feedback.

In some embodiments, the first processor may control the pulse generator module to emit electrical pulses based on the sacral neuromodulation signal, to form the nerve stimulation and suppress the urinary urgency episode.

The pulse generator module refers to an actual execution hardware configured to apply the sacral neuromodulation signal to the target user's body, which may include electrodes, cables, peripherals, pulse generators, etc., and is part of the target terminal device.

When the sacral neuromodulation signal is triggered, the first processor immediately controls the pulse generator module to emit the electrical pulses according to preset parameters (e.g., frequency 14 Hz, pulse width 210 μs, intensity slightly below the threshold intensity of the intraoperative motor and sensory responses).

In some embodiments, the first processor may collect contact impedance through a four-contact electrode array; based on the contact impedance, perform self-correction on the stimulation contacts, including: detecting an electrode contact state based on the contact impedance to control the pulse generator module to dynamically switch the stimulation contacts.

The contact impedance refers to an additional impedance, which impedes current flow, formed between the electrode and a contact surface of the sphincter receiving neuromodulation in the target user. It may be used to judge the contact quality between the electrode and the contact surface of the sphincter.

The self-correction refers to a process where the system, without user intervention, automatically determines whether the current stimulation contact has an abnormality based on the real-time collected contact impedance of the electrode, and dynamically adjusts the stimulation contacts.

The stimulation contacts refer to a part of the electrode in the target terminal device that is in direct contact with the muscle or tissue of the target user and applies electrical pulses. The stimulation contact may be a single metal contact point or area on the electrode array.

A four-contact electrode array refers to a single electrode device surface integrating four independently addressable stimulation contacts, used for directional stimulation of different nerve branches.

In some embodiments, by applying a low-voltage test current to the stimulation contacts and measuring the voltage response, the contact impedance is calculated.

The electrode contact states include normal contact, abnormal contact, etc.

In some embodiments, the first processor may detect the electrode contact state based on contact impedance to control the pulse generator module to dynamically switch stimulation contacts. For example, if the contact impedance exceeds a preset impedance range, the electrode contact state is determined as abnormal. When the contact state is abnormal, the pulse generator module automatically switches to a stimulation contact with normal impedance. The preset impedance range may be predefined manually.

In some embodiments of the present disclosure, by collecting the contact impedance to detect the electrode contact state and dynamically switching the stimulation contacts, the self-correction of the stimulation position is achieved. This improves the reliability of electrode contact and the accuracy of stimulation position, avoiding abnormal stimulation or improper stimulation caused by poor contact, user movement, or accidental contact.

In some embodiments, the first processor may dynamically adjust the stimulation parameters of the sacral neuromodulation signal based on a user activity state and environmental data; and, determine, based on the user activity state and correlation analysis results performed on the urinary flow rate, the bladder pressure, and the external anal sphincter electromyogram signal, whether the external anal sphincter electromyogram signal is a false signal; in response to determining that the external anal sphincter electromyogram signal is not the false signal, determine an advance stimulation time based on the urinary flow rate, the bladder pressure, and the external anal sphincter electromyogram, and control the pulse generator module to emit the electrical pulses at the advance stimulation time.

The user activity state may include a daytime working state, a nighttime sleep state, and an exercise state, which may be collected in real time through sensors (e.g., speed sensors) or medical monitoring devices (e.g., sleep monitors, electrocardiogram

The environmental data may include temperature and humidity data of the user's surrounding environment, which may be monitored and obtained through sensors (e.g., temperature and humidity sensors).

The stimulation parameters of the sacral neuromodulation signal include the frequency, the pulse width, and the intensity of the stimulation signal. Specific details may be found in the description related to FIG. 1.

In some embodiments, the first processor may establish a second preset table based on the user activity state and the environmental data, and then dynamically adjust the stimulation parameters of the sacral neuromodulation signal by querying the second preset table. The second preset table includes the correspondence between the user activity state, the environmental data, and the stimulation parameters. For example, during the daytime working state, the stimulation parameters are moderate to balance treatment efficacy and comfort; during the nighttime sleep state, neural sensitivity is higher, set gentler stimulation parameters to avoid disturbing sleep; during the exercise state, appropriately increase stimulation frequency and intensity to adapt to neural excitement, and appropriately decrease pulse width to ensure comfort, etc.

In some embodiments, the second preset table may be constructed based on the treatment records of the historical users with good treatment effects (e.g., the stimulation effectiveness ≥80%). For example, the second preset table is constructed based on the large number of treatment records of the historical users with good treatment effects. Each record includes the user's different activity states during treatment, the environmental data corresponding to each activity state, and the stimulation parameters of the actually used sacral neuromodulation signal. Fill a table with the data to construct the second preset table.

The correlation analysis refers to analyzing the statistical dependence between parameters, such as, whether a rise in the urinary flow rate is always accompanied by a rise in the bladder pressure.

In some embodiments, the first processor may determine whether the external anal sphincter electromyogram signal is the false signal based on the results the correlation analysis performed on the urinary flow rate, the bladder pressure, and the external anal sphincter electromyogram signal, and the user activity state. For example, if there is fluctuation in the external anal sphincter electromyogram signal, but no rise in the urinary flow rate is detected within a preset time window (or the rise amplitude is below a preset amplitude threshold), or no change in the bladder pressure is detected (or the pressure change value is below a preset change threshold), or the user is currently in an activity state (e.g., the exercise state, etc.) prone to generating the false signal, the first processor may determine the external anal sphincter electromyogram signal as the false signal.

The false signal refers to a signal unrelated to urination but similar to urinary urgency-related signals, such as, a electromyogram signal similar to urinary urgency-related signals caused by the user feeling skin discomfort rather than urinary urgency.

The advance stimulation time refers to a time difference by which neuromodulation is delivered in advance relative to an estimated time point of the urinary urgency episode. A larger value of the advance stimulation time indicates earlier intervention of the neuromodulation; a smaller value indicates that the intervention timing is closer to the estimated time point of the urinary urgency episode.

In some embodiments, in response to determining that the external anal sphincter electromyogram signal is not the false signal, the first processor may determine the advance stimulation time based on the quantitative association of parameters such as the urinary flow rate, the bladder pressure, and the electromyography (EMG) signal. For example, when the urinary flow rate surges, the bladder pressure continuously rises, and the EMG electromyogram amplitude exceeds a target reference interval, it indicates that the user is closer to a true urinary urgency or voiding episode state. At the point, the advance stimulation time should be larger, meaning that neuromodulation intervenes earlier to avoid urine leakage or loss of voiding control, improving stimulation success rate. That is, the advance stimulation time is positively correlated with the urinary flow rate, the bladder pressure, and EMG electromyogram.

In some embodiments of the present disclosure, by dynamically adjusting the stimulation parameters of the sacral neuromodulation signal, accidental or inadvertent activation of neuromodulation signals by users is prevented. Since different physiological states of the human body affect electrical signals, by distinguishing these states and dynamically adjusting the EMG threshold (i.e., the external anal sphincter electromyogram signal trigger threshold) used to trigger stimulation, over-stimulation or under-stimulation caused by misjudgment is effectively avoided, making stimulation control better align with the user's actual needs.

In some embodiments, the first processor may obtain the external anal sphincter electromyogram signal amplitude reference interval and signal features of similar users, where the similar users are other users with training features that are similar to those of a current user (e.g., the target user); based on the external anal sphincter electromyogram signal amplitude reference interval and the signal features of the similar users, further determine whether the external anal sphincter electromyogram signal is the false signal.

The training features that are similar to those of the current user mean that a similarity between the training features of the other user and the training features of the current user is greater than a preset similarity threshold. In some embodiments, the first processor may extract the training features of the target user and vectorize the training features, calculate similarity of the training features of the other users in a historical training sample library with the training features of the target user using cosine similarity algorithms or other manners, screen out other users with similarity higher than the preset similarity threshold as the similar users, and then obtain the external anal sphincter electromyogram signal amplitude reference interval of the similar users.

The signal features refer to characteristic parameters of the external anal sphincter electromyogram signal, which may include signal frequency, signal duration, signal waveform, etc. In some embodiments, the first processor may extract signal features based on the acquired external anal sphincter electromyogram signal using feature extraction manners such as filtering, threshold segmentation, and spectral analysis.

In some embodiments, the first processor may compare the external anal sphincter electromyogram signal amplitude reference interval and the signal features of the similar users with a preset normal reference interval and normal signal features; determines the signal as the false signal in response to determining that abnormalities exist (e.g., no intersection or intersection length less than a preset length threshold between the external anal sphincter electromyogram signal amplitude reference interval of the similar users and the preset normal reference interval, abnormal signal frequency or duration, signal waveform exhibiting active contraction characteristics or behavior-induced characteristics, etc.).

The active contraction characteristics refer to electromyogram signal features generated when the user consciously controls the muscle (e.g., following doctor's instructions, performing Kegel exercises, etc.). It is usually characterized by a regular waveform, strong repeatability, stable amplitude, relatively controllable duration, overall uniform signal, and easily distinguishable from reflexive irregular signals.

The behavior-induced characteristics refer to electromyogram signal features generated by the muscle's short-term response caused by the user's non-urination-related behaviors (e.g., coughing, sneezing, emotional excitement, posture change, etc.). It is usually characterized by sudden, short, high-amplitude, but extremely short-duration fluctuations, an absence of the sustained and characteristic preparatory phase preceding voiding contractions.

In some embodiments of the present disclosure, by comparing the electromyogram signal amplitude reference interval and signal features of similar users to identify whether the external anal sphincter electromyogram signal of the target user is a false signal, the triggering timing of sacral neuromodulation is optimized, enhancing the therapeutic effect for the patient.

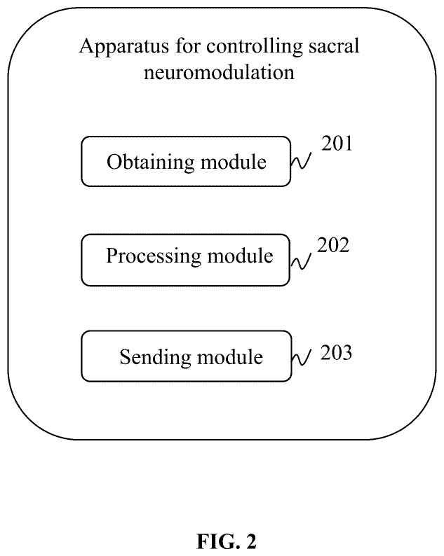

FIG. 2 is a structural schematic diagram of an apparatus for controlling sacral neuromodulation according to some embodiments of the present disclosure. As shown in FIG. 2, the apparatus for controlling sacral neuromodulation includes an obtaining module 201, a processing module 202, and a sending module 203.

The obtaining module 201 is configured to obtain historical physiological parameter information of a target user and real-time physiological parameter information of the target user, and obtain a preset biological neural stimulation signal generation model matching identification information.

The processing module 202 is configured to: process the real-time physiological parameter information of the target user to generate to-be-monitored user data with the identification information, wherein the identification information is used to characterize a target item currently to be detected for the target user; process the preset biological neural stimulation signal generation model based on historical physiological parameter signals of the target user to generate a target biological neural stimulation signal generation model, wherein the historical physiological parameter signals of the target user include training samples corresponding to the target item currently to be detected for the target user; process the real-time physiological parameter information of the target user based on the target biological neural stimulation signal generation model to generate a real-time external anal sphincter electromyogram signal, wherein the real-time external anal sphincter electromyogram signal includes an external anal sphincter electromyogram signal amplitude, the external anal sphincter electromyogram signal amplitude including a target external anal sphincter diastole amplitude and a target external anal sphincter systole amplitude; and process the external anal sphincter electromyogram signal amplitude based on a preset processing rule to generate a sacral neuromodulation signal, wherein a preset threshold is set based on the real-time physiological parameter information of the target user.

The sending module 203 is configured to send the sacral neuromodulation signal to a target terminal device, so that the target terminal device operates on the target user.

FIG. 3 is a structural schematic diagram of an electronic device according to some embodiments of the present disclosure.

As shown in FIG. 3, an electronic device 3 includes a first processor 300, a memory 301, a bus 302, and a communication interface 303. The first processor 300, the communication interface 303, and the memory 301 are connected via the bus 302. The memory 301 stores a computer program executable on the first processor 300. When the first processor 300 runs the computer program, it executes the method for controlling sacral neuromodulation provided in any of the foregoing embodiments of the present disclosure.

The memory 301 may include high-speed random access memory (RAM) and may also include non-volatile memory, such as at least one disk storage. Communication connection between the system network element and at least one other network element is realized through at least one communication interface 303 (which may be wired or wireless), and the Internet, wide area network, local network, metropolitan area network, etc., may be used.

The bus 302 may be an industry standard architecture (ISA) bus, a peripheral component interconnect (PCI) bus, an extended industry standard architecture (EISA) bus, etc. The bus may be divided into an address bus, a data bus, a control bus, etc. The memory 301 is configured to store the computer program. After receiving an execution instruction, the first processor 300 executes the computer program. The method for controlling sacral neuromodulation disclosed in any of the foregoing embodiments of the present disclosure may be applied to the first processor 300 or implemented by the first processor 300.

The first processor 300 may be an integrated circuit chip with signal processing capabilities. In the implementation process, each step of the above method may be completed by the integrated logic circuit of the hardware in the first processor 300 or instructions in the form of software. The aforementioned first processor 300 may be a general-purpose processor, including a central processing unit (CPU), network processor (NP), etc, may also be a digital signal processor (DSP), application specific integrated circuit (ASIC), field-programmable gate array (FPGA) or other programmable logic devices, discrete gates or transistor logic devices, discrete hardware components. It may implement or execute the various methods, steps, and logic block diagrams disclosed in the embodiments of the present disclosure. A general-purpose processor may be a microprocessor or the processor may be any conventional processor, etc. The steps of the method disclosed in conjunction with the embodiments of the present disclosure may be embodied as being executed and completed by a hardware decoding processor, or executed and completed by a combination of hardware and software modules in the decoding processor. The software module may be located in a mature storage medium in the art such as random access memory, flash memory, read-only memory, programmable read-only memory, electrically erasable programmable memory, registers, etc. The storage medium is located in the memory 301. The first processor 300 reads information in the memory 301 and, in combination with its hardware, completes the steps of the above method.

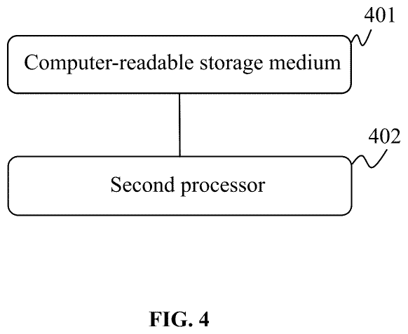

FIG. 4 is a schematic diagram of a storage medium according to some embodiments of the present disclosure.

As shown in FIG. 4, a computer-readable storage medium 401 stores a computer program. When the computer program is read and run by a second processor 402, it implements the method for controlling sacral neuromodulation as described above.

The technical solution of the embodiments of the present disclosure, or the part contributing to the prior art, or all or part of the technical solution, may be embodied in the form of a software product. The computer software product is stored in a storage medium, including several instructions for causing an electronic device (which may be an air conditioner, refrigeration device, personal computer, server, or network device, etc.) or a processor to execute all or part of the steps of the method described in the embodiments of the present disclosure. The aforementioned storage medium includes various media that may store program codes, such as U disks, mobile hard disks, read-only memory (ROM), random access memory (RAM), magnetic disks, or optical disks.

The computer-readable storage medium provided by the above embodiments of the present disclosure shares the same inventive concept as the method for controlling sacral neuromodulation provided by the embodiments of the present disclosure, and it delivers the same beneficial effects as the method adopted, executed, or implemented by the stored application.

The embodiments of the present disclosure provide a computer program product, comprising a computer program that, when executed by a third processor, implements the aforementioned method.

The computer program product provided by the embodiments of the present disclosure shares the same inventive concept as the method for controlling sacral neuromodulation provided by the embodiments of the present disclosure, and it delivers the same beneficial effects as the method adopted, executed, or implemented by the stored application.

Furthermore, certain features, structures, or characteristics in one or more embodiments of the present disclosure may be appropriately combined.

It should be noted that if there are inconsistencies or conflicts between the descriptions, definitions, and/or terminology used in the supplementary materials of the present disclosure and those in the present disclosure, the descriptions, definitions, and/or terminology used in the present disclosure shall prevail.

What is claimed is:

1. A method for controlling sacral neuromodulation, comprising:
obtaining historical physiological parameter information of a target user and real-time physiological parameter information of the target user;
processing the real-time physiological parameter information of the target user to generate to-be-monitored user data with identification information, wherein the identification information is used to characterize a target item currently to be detected for the target user;
obtaining a preset biological neural stimulation signal generation model matching the identification information;
processing the preset biological neural stimulation signal generation model based on historical physiological parameter signals of the target user to generate a target biological neural stimulation signal generation model, wherein the historical physiological parameter signals of the target user include training samples corresponding to the target item currently to be detected for the target user;
processing the real-time physiological parameter information of the target user based on the target biological neural stimulation signal generation model to generate a real-time external anal sphincter electromyogram signal, wherein the real-time external anal sphincter electromyogram signal includes an external anal sphincter electromyogram signal amplitude, the external anal sphincter electromyogram signal amplitude includes a target external anal sphincter diastole amplitude and a target external anal sphincter systole amplitude; and
processing the external anal sphincter electromyogram signal amplitude based on a preset processing rule to generate a sacral neuromodulation signal, wherein a preset threshold is set based on the real-time physiological parameter information of the target user.

2. The method according to claim 1, wherein processing the preset biological neural stimulation signal generation model based on the historical physiological parameter signals of the target user to generate the target biological neural stimulation signal generation model includes:
performing feature extraction on the historical physiological parameter signals of the target user to generate an original feature library, wherein the original feature library includes discrete variables and continuous variables;
processing the historical physiological parameter signals of the target user based on the discrete variables to generate an initial training set; and
processing the initial training set based on the continuous variables to generate a target training set, wherein the target training set includes a plurality of external anal sphincter electromyogram signal amplitude reference intervals.

3. The method according to claim 2, wherein processing the preset biological neural stimulation signal generation model based on the historical physiological parameter signals of the target user to generate the target biological neural stimulation signal generation model further includes:
processing the target training set to generate multiple continuous variables matching the identification information;
processing the multiple continuous variables based on a preset function to generate a target external anal sphincter electromyogram signal amplitude reference interval, wherein the target external anal sphincter electromyogram signal amplitude reference interval is a preset reference range matching the target user; and
generating the target biological neural stimulation signal generation model based on the target external anal sphincter electromyogram signal amplitude reference interval.

4. The method according to claim 1, wherein processing the real-time physiological parameter information of the target user to generate the to-be-monitored user data with the identification information includes:
processing the real-time physiological parameter information of the target user to generate user attribute information, wherein the user attribute information includes the target item currently to be detected for the target user;
performing feature extraction on the real-time physiological parameter information of the target user to generate a target feature library; and
processing the user attribute information based on the target feature library to generate the to-be-monitored user data with the identification information.

5. The method according to claim 2, wherein processing the real-time physiological parameter information of the target user based on the target biological neural stimulation signal generation model to generate the real-time external anal sphincter electromyogram signal includes:
processing the target training set based on the target biological neural stimulation signal generation model to generate a preset external anal sphincter electromyogram signal dataset;
processing the preset external anal sphincter electromyogram signal dataset to obtain stimulation features and a correlation co-occurrence frequency;
processing the stimulation features and the correlation co-occurrence frequency to generate correlation matrix information;
generating a prior knowledge graph for characterizing the real-time external anal sphincter electromyogram signal based on the correlation matrix information; and
processing the real-time physiological parameter information of the target user based on the prior knowledge graph to generate the real-time external anal sphincter electromyogram signal.

6. The method according to claim 5, wherein processing the stimulation features and the correlation co-occurrence frequency to generate the correlation matrix information, includes:
a first calculation formula for calculating the correlation co-occurrence frequency, the first calculation formula being:
$${P}_{ij} = \frac{{C}_{ij}}{{C}_{j}} ;$$
where Cᵢⱼ represents a count of times concept i and concept j co-occur at a report level, Cⱼ represents a total count of times concept j appears, and Pᵢⱼ represents a frequency of concept i appearing when concept j appears; and
a second calculation formula for calculating the correlation matrix information, the second calculation formula being:
$${A}_{ij} = \{ \begin{matrix}1 , & if {P}_{ij} \geq τ \\ 0 , & if {P}_{ij} < τ\end{matrix} ;$$
where τ represents a co-occurrence frequency threshold, and if Pᵢⱼ is greater than or equal to τ, it is considered that there is a correlation from concept i to concept j, otherwise there is no correlation.

7. The method according to claim 1, wherein processing the external anal sphincter electromyogram signal amplitude based on the preset processing rule to generate the sacral neuromodulation signal includes:
processing the real-time external anal sphincter electromyogram signal to generate an electromyogram signal feature vector;
processing the electromyogram signal feature vector based on the target biological neural stimulation signal generation model to generate a classification result, wherein the classification result is set based on the external anal sphincter electromyogram signal amplitude; and
generating the sacral neuromodulation signal if the classification result is that the external anal sphincter electromyogram signal amplitude is greater than the preset threshold.

8. An apparatus for controlling sacral neuromodulation, comprising:
an obtaining module configured to:
obtain historical physiological parameter information of a target user and real-time physiological parameter information of the target user; and
obtain a preset biological neural stimulation signal generation model matching identification information;
a processing module configured to:
process the real-time physiological parameter information of the target user to generate to-be-monitored user data with the identification information, wherein the identification information is used to characterize a target item currently to be detected for the target user;
process the preset biological neural stimulation signal generation model based on historical physiological parameter signals of the target user to generate a target biological neural stimulation signal generation model, wherein the historical physiological parameter signals of the target user include training samples corresponding to the target item currently to be detected for the target user;
process the real-time physiological parameter information of the target user based on the target biological neural stimulation signal generation model to generate a real-time external anal sphincter electromyogram signal, wherein the real-time external anal sphincter electromyogram signal includes an external anal sphincter electromyogram signal amplitude, the external anal sphincter electromyogram signal amplitude including a target external anal sphincter diastole amplitude and a target external anal sphincter systole amplitude; and
process the external anal sphincter electromyogram signal amplitude based on a preset processing rule to generate a sacral neuromodulation signal, wherein a preset threshold is set based on the real-time physiological parameter information of the target user; and
a sending module configured to:
send the sacral neuromodulation signal to a target terminal device, so that the target terminal device operates on the target user.

9. An electronic device, comprising:
a first processor; and
a memory for storing executable instructions of the first processor;
wherein the first processor is configured to implement the method for controlling sacral neuromodulation according to claim 1 by executing the executable instructions.

10. A non-transitory computer-readable storage medium comprising a computer program stored thereon, wherein the computer program, when executed by a second processor, implements the method for controlling sacral neuromodulation according to claim 1.
